(19) United States
(12) Patent Application Publication
(10) Pub. No.: US 2008/0021521 A1
(43) Pub. Date: Jan. 24, 2008

(54) Implantable Medical Device Communication System

(75) Inventors: Yogendra A. Shah, Blaine, MN (US); Sasidhar Vajha, Brooklyn Park, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 11/751,966

(22) Filed: May 22, 2007

Related U.S. Application Data
(60) Provisional application No. 60/831,509, filed on Jul. 18, 2006.
(60) Provisional application No. 60/887,069, filed on Jan. 29, 2007.

(51) Int. Cl. A61N 1/02

(52) U.S. Cl. 607/60

(57) ABSTRACT

A medical communication system for providing remote communications with an active implantable medical device. In one embodiment, a medical communication system includes an active implantable medical device (AIMD) that is configured to transmit and receive a wireless signal from within a human body, and a non-implantable programmer that includes a retrodirective antenna. The programmer is configured to scan in multiple directions for signals received from the AIMD and to identify the direction of the signal having the highest signal power.

### RELATED APPLICATIONS

This application claims priority to provisional U.S. patent application 60/831,509, filed Jul. 18, 2006; and provisional U.S. patent application 60/887,069, filed Jan. 29, 2007.

### FIELD OF THE INVENTION

The invention relates to medical communications systems, and more particularly, to the communication of information between an active implantable medical device and a remote location.

### BACKGROUND OF THE INVENTION

A variety of active implantable medical devices (AIMD) are used to provide medical therapy to patients, where an AIMD incorporates some type of electronics or electronic signal processing to deliver a medical therapy. One example of a type of active implantable medical device is a cardiac rhythm management (CRM) device. CRM devices may include, for example, pacemakers and implantable cardioverter defibrillators (ICD). These devices generally provide medical treatment to a patient having a disorder relating to the pacing of the heart, such as bradycardia or tachycardia. For example, a patient having bradycardia may be fitted with a pacemaker, where the pacemaker is configured to monitor the patient's heart rate and to provide an electrical pacing pulse to the cardiac tissue if the heart fails to naturally produce a heart beat at a sufficient rate. By way of further example, a patient may have an ICD implanted to provide an electrical shock to the patient's heart if the patient experiences fibrillation.

Certain AIMDs have sensors that are configured to sense a physical parameter of the patient's body. Some AIMDs also have electronic circuitry that is capable of recording data that is representative of a patient's physical condition, such as data recorded from sensors or data relating to the patient's heart rate and therapy delivered. AIMDs may further be configured to receive instructions from an external source to modify and control the operation of the AIMD. For example, a physician may transmit instructions from an external device to an implanted medical device within a patient to change the therapy administered to the patient in response to the physician's analysis of information received about the patient's condition.

In a typical configuration, an AIMD is provided with an antenna for communicating by telemetry with a device outside of the patient's body. In one case, the device outside of the patient's body is a wand that is held against or near the patient's body in the vicinity of the implanted device. The wand is conventionally magnetically or inductively coupled to the IMD and is wired to a programmer and recorder module that receives and analyzes the information from the implanted device and that may provide an interface for a person such as a physician to review the information. A wired connection between a wand and a programmer has the advantage of providing a clear signal with high gain, as well as security of transmission. However, a wired connection is often inconvenient, because the wire can get caught on equipment or interfere with the movement of people and other equipment in the vicinity. The wire may also become tangled and limits the portability and mobility of the programmer.

One consideration associated with the transmission of information by way of telemetry from a device implanted within a patient's body is the allowable exposure of the patient's body tissue to electromagnetic radiation. For example, it is important that both the power and the frequency of the transmission to and from the implantable medical device do not cause appreciable tissue heating. It is also important that the electromagnetic radiation not be mutagenic or otherwise harmful to the patient.

An additional consideration associated with the transmission of information from a device implanted within a patient's body is the potential for interference with the signal. Wireless signals are often transmitted from implantable medical devices in hospitals or other medical care facilities that tend to have a significant number of other wireless devices present. These environments tend to be prone to interference as a consequence of the number of other wireless signals being transmitted in the same or adjacent frequency bands. The signal from the AIMD should therefore be robust to interference.

There is also a concern regarding the incompatibility of the available frequencies from one country to the next. Furthermore, some countries may not have frequency bands allocated for medical telemetry at all. For example, medical device communication systems may operate in the frequency ranges of 402 to 405 MHz, or alternatively 902 to 928 MHz, in the United States and Canada, and in the range of 869.7 to 870 MHz in Europe. These differences in allocated frequency bands render a device used in one geographic location incompatible for use in another geographic location. The incompatibility of frequencies creates a risk that a patient who has traveled from one country to another will not receive the medical therapy or device programming that is required, and also creates difficulties for manufacturers of medical communication systems who have to design different devices for each geographic region rather than offering a product that can work anywhere.

Yet another consideration is the size of the antenna required to be part of the AIMD. The nature of the task of implanting a medical device within a person is such that it is desired that the device be as small as possible. The size of the antenna can be a significant portion of the overall size of the implantable device.

A further consideration is the ability to transmit the information received from the AIMD to a remote location. In some cases, a patient may live in an area where a physician or other trained person is not available to review data received from the implantable medical device and to determine the appropriate medical therapy to deliver or proper control of the implantable device. Furthermore, some patients may be located in areas where traditional means of communication, such as by telephone or over the Internet, are not available.

Some wireless communications systems for medical devices may rely on line of sight between the point of transmission and the point of reception. In a crowded medical environment, however, the line of sight may be degraded due to the locations of people and objects. Various people may walk in and out of the line of sight, causing the transmission to be dropped or halted, possibly while a medical procedure or surgery is being performed. Therefore, it is desired that a wireless communications system not be dependent upon maintaining line of sight transmission.

There is also a need with implantable medical devices to monitor the performance of the device during the surgical procedure in which it is implanted. In some cases, a physician may be present in the surgical suite who is trained and competent to analyze the data transmitted from the device during the surgical procedure. In other cases, the physician who can analyze the data is not located on site, and the data must be transmitted to a remote location for analysis and review.

Improved communications of signals to and from implantable medical devices are needed.

### SUMMARY OF THE INVENTION

A medical communication system for providing remote communications with an active implantable medical device is disclosed. In one aspect, a medical communication system includes an active implantable medical device (AIMD) that is configured to transmit and receive a wireless signal from within a human body, and a non-implantable programmer that includes a retrodirective antenna. The programmer is configured to scan in multiple directions for signals received from the AIMD and to identify the direction of the signal having the highest signal power.

In another aspect, a medical communication system includes an active implantable medical device (AIMD) that is configured to transmit a wireless signal and a programmer that has a retrodirective antenna that is configured to receive the wireless signal from the AIMD and to transmit a corresponding signal. The communication system further includes a local site repeater that is configured to receive the signal from the programmer and to transmit a corresponding signal, a local ground station that is configured to receive the signal from the site repeater and to transmit a corresponding signal, and a space-based satellite that is configured to receive the signal from the local ground station and to transmit a corresponding signal to a remote ground station. The remote ground station is configured to receive the signal from the space-based satellite and to transmit a corresponding signal. Furthermore, the system includes a remote site repeater that is configured to receive the signal from the remote ground station and to transmit a corresponding signal, and a remote device that is configured to receive the signal from the remote site repeater and to provide an interface to the signal.

The invention may be more completely understood by considering the detailed description of various embodiments of the invention that follows in connection with the accompanying drawings.

### BRIEF DESCRIPTION OF THE DRAWINGS

FIG. 1 is a medical device communication system for transmitting information from an implantable medical device to a remote location constructed according to the principles of the present invention.

FIG. 2 is a retrodirective antenna array for use with the medical device communication system of FIG. 1.

FIG. 3 is a plan view of a surgical suite illustrating the behavior of a transmitted signal where there is line of sight from an implantable medical device to a programmer/recorder/monitor.

FIG. 4 is a plan view of a surgical suite illustrating the behavior of a transmitted signal where there is not line of sight from an implantable medical device to a programmer/recorder/monitor.

FIG. 5 is a schematic view of an implantable medical device that is implanted in a patient.

FIG. 6 is an exploded view of an aperture coupled circularly polarized retrodirective antenna element.

FIG. 7 is an alternative embodiment of a medical device communication system having a wand in communication with an implantable medical device.

FIG. 8 is a plan view of a transmission from a wireless wand to a programmer where line of sight is available.

FIG. 9 is a plan view of a transmission from a wireless wand to a programmer where line of sight is not available.

FIG. 10 is a functional block diagram illustrating modules that implement a wireless wand communication system.

FIG. 11 illustrates a programmer with a phased array of patch antennas for use in communication with a wireless wand.

FIGS. 12-13 illustrate antenna radiation patterns generated at selected scan angles around a programmer for an implantable medical device.

FIG. 14 is a flow chart illustrating an algorithm that may be carried out by a programmer for communicating with an implantable medical device.

While the invention may be modified in many ways, specifics have been shown by way of example in the drawings and will be described in detail. It should be understood, however, that the intention is not to limit the invention to the particular embodiments described. On the contrary, the intention is to cover all modifications, equivalents, and alternatives following within the scope and spirit of the invention as defined by the claims.

### DETAILED DESCRIPTION OF THE INVENTION

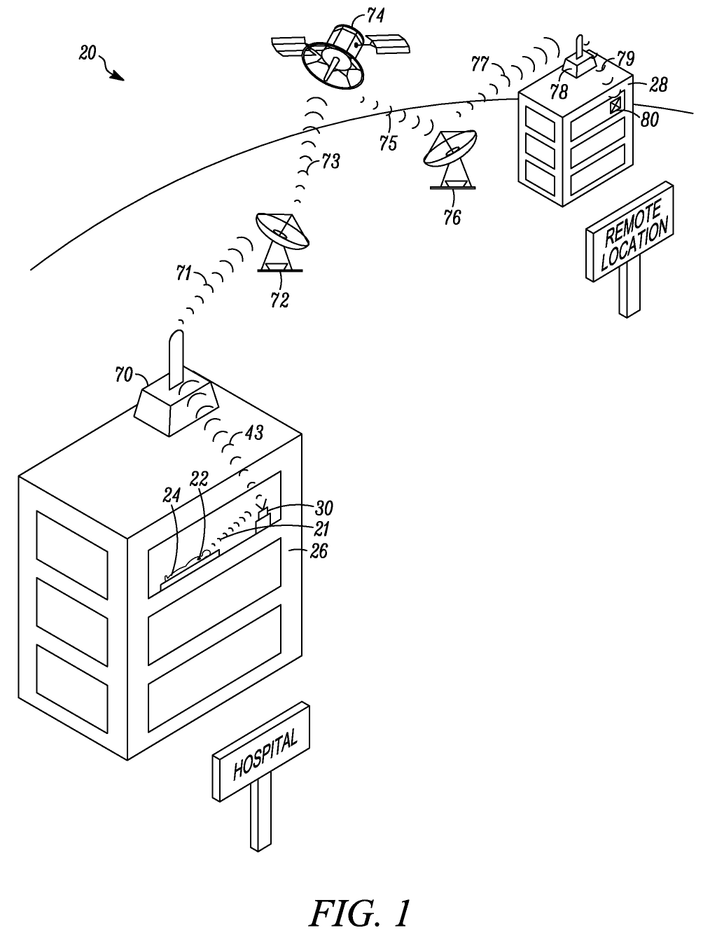

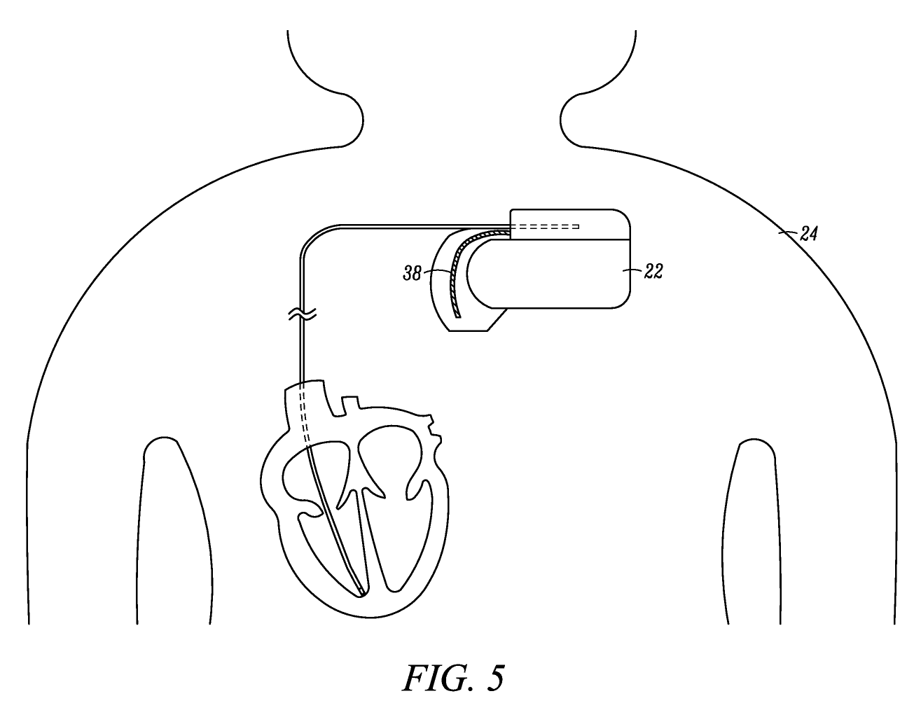

An embodiment of the invention is depicted in FIG. 1. Medical device communication system 20 is configured to transmit a signal having encoded information from an active implanted medical device (AIMD) 22 that is in a patient 24 who is in a medical facility 26 to a remote location 28. In the embodiment of FIG. 1, AIMD 22 communicates by a telemetric signal 21 to a programmer/recorder/monitor (PRM) 30 that is located within range of the transmission signal 21 from the AIMD 22. An embodiment of AIMD 22 is depicted in FIG. 5. In one usable embodiment, PRM 30 is located in the same room as the patient 22. In another embodiment, PRM 30 is located in an adjacent room to patient 24. In yet another embodiment, the PRM 30 is located anywhere within the range of the transmission from the AIMD 22. In some embodiments, the AIMD is capable of transmitting a signal 21 about 3 meters (10 feet). In other embodiments, the AIMD is capable of transmitting signal 21 about 10 meters (30 feet). In yet other embodiments, the AIMD is capable of transmitting a signal 21 about 23 meters (75 feet). The transmission of signal 21 from AIMD 22 to PRM 30 is preferably conducted at high frequency. In one embodiment, the transmission signal frequency 21 is equal to or greater than 1 GHz. In another embodiment, the transmission signal frequency 21 is equal to or greater than 2 GHz. In a further embodiment, the transmission signal frequency 21 is 2.4 to 2.5 GHz. In yet another embodiment, the transmission signal frequency 21 is 2.4835 to 2.5 GHz. In some embodiments, the transmission signal frequency 21 is in the S band of about 2 to 4 GHz. In some other embodiments, the transmission signal frequency 21 is in the C-band of about 4 to 8 GHz. In yet other embodiments, the transmission signal frequency 21 is in the X-band of about 8 to 12 GHz.

All signal transmissions in the medical device communication system 20 are bidirectional. Signals can be transmitted in the direction from AIMD 22 to a remote location 28, and signals can also be transmitted in the direction from the remote location 28 to the AIMD 22. For ease of description, however, the transmission of signals will generally be described herein as occurring in the direction from the AIMD 22 to the remote location 28. It will be appreciated that such description applies equally to transmissions in the alternate direction, namely, in the direction from the remote location 28 to the AIMD 22.

There are many usable embodiments of PRM 30. PRM 30 may have various features including electronic data storage features such as a disk drive and/or a hard disk drive, and data interfacing features such as a monitor and/or a printer. In one embodiment, PRM 30 includes programming, recording, and monitoring functions, and in other embodiments PRM 30 includes less than all of these functions or features. PRM 30 may also be called a programmer 30. In some embodiments, there is one PRM 30 that receives a signal 21 from one AIMD 22. In other embodiments, one PRM 30 is configured to receive signals from multiple AIMDs 22 located within the medical facility 26.

AIMD 22 and PRM 30 each have an antenna for receiving and transmitting electromagnetic signals. There are many usable embodiments of the antenna used with AIMD 22. For example, the AIMD antenna can be a monopole antenna, a dipole antenna, or a planar antenna, among others. In one embodiment, the AIMD 22 has a dipole antenna 38, where the dipole antenna 38 is characterized by a length that is optimized for the wavelength of the signal to be received and transmitted. An AIMD 22 having a dipole antenna 38 is depicted in FIG. 5. In one embodiment, the dipole antenna 38 is constructed from a conductive material that has a length dimension that is significantly larger than its other dimensions, such as a wire. In one embodiment, the dipole antenna has a length that is about ¼ the wavelength of the signal 21. The AIMD antenna 38 is configured to transmit an omni-directional signal 21 that travels in all directions or substantially all directions away from the antenna with equal signal strength. The AIMD antenna 38 is also configured in some embodiments to transmit a pilot signal to initiate communications with PRM 30. Signals transmitted by the AIMD antenna 38 can be modulated using any of various modulation techniques, such as amplitude shift keying (ASK, e.g., on-off keying (OOK)), binary phase-shift keying (BPSK), quadrature phase-shift keying (QPSK), and Gaussian phase-shift keying (GPSK).

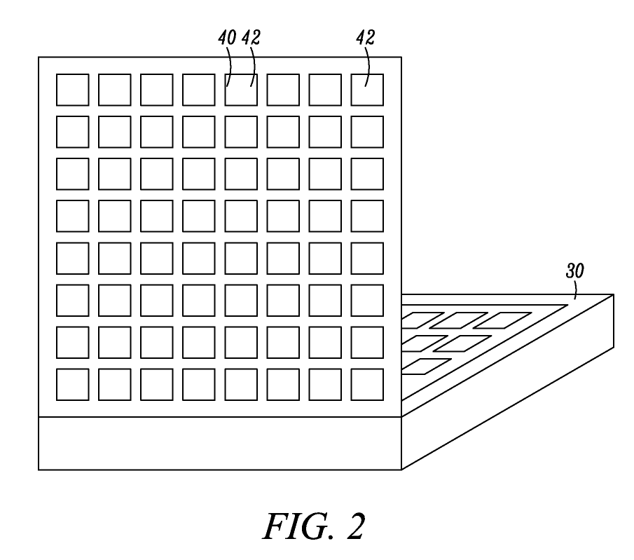

In one embodiment, PRM 30 has a retrodirective antenna 40. A PRM 30 having a retrodirective antenna 40 is depicted in FIG. 2. A retrodirective antenna is generally an antenna that is capable of receiving a pilot wave from another antenna located away from it, determining the direction of the pilot wave source, and locking to the direction of the pilot wave source. In one embodiment, the PRM 30 is configured like a typical laptop computer, having a screen connected to a main body of the computer, and having an input device such as a keyboard on the body of the computer. In this type of embodiment, the retrodirective antenna is positioned on the back of the screen as shown in FIG. 2.

In one embodiment the retrodirective antenna 40 is a phased array of patch antennas 42. In the embodiment of FIG. 2, the number of patch antennas ranges from 4 to 100. In one embodiment, there are sixty-four patch antennas 42 that make up the retrodirective antenna 40, where the sixty-four patch antennas 42 are arranged in an array that is eight patch antennas wide by eight patch antennas tall. Patch antennas 42 have the advantages that they are light weight and inexpensive, and also can be mounted on a flat surface of PRM 30. Each patch antenna 42 generally has a range of about 0 to 6 dB in gain, and by forming an array of patch antennas 42, the gain can be increased. The patch antennas 42 can also be conformed to the surface of PRM 30 to take up less area and minimize the volume of the PRM 30.

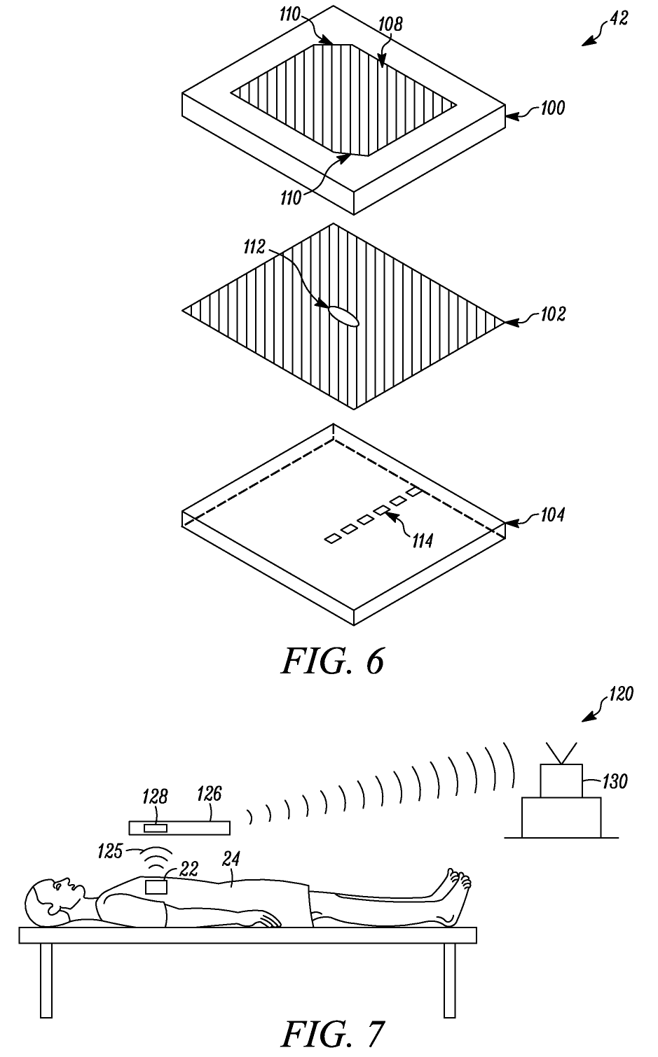

An example of a patch antenna 42 is depicted in FIG. 6. The antenna 42 of FIG. 6 is aperture coupled and circularly polarized using perturbations. Patch antenna 42 is formed from a substrate 100, a ground plane 102, and an active substrate/feed line 104. The antenna substrate 100 depicted in FIG. 6 has a circularly polarized patch radiator 108, which includes perturbations intended for circular polarization 110. Ground plane 102 has an aperture 112. Active substrate 104 includes a microstrip feed line 114. In one embodiment, the microstrip feed line 114 is characterized as having a 50 Ohm resistance.

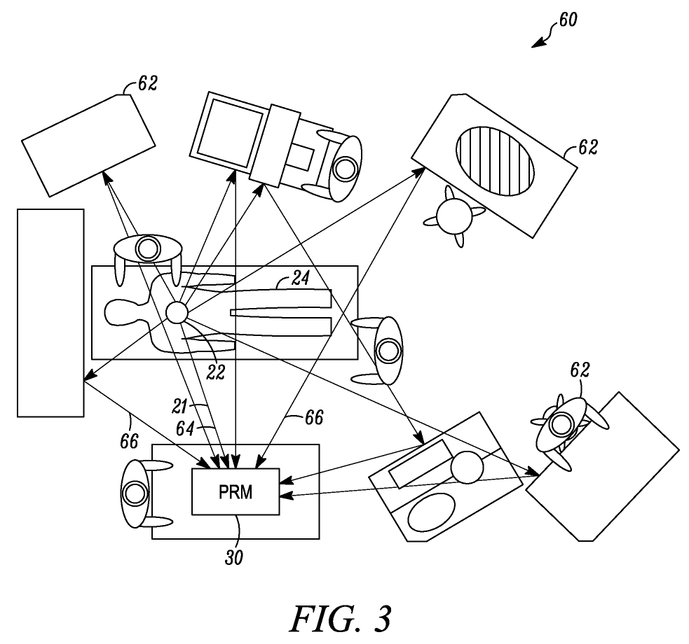

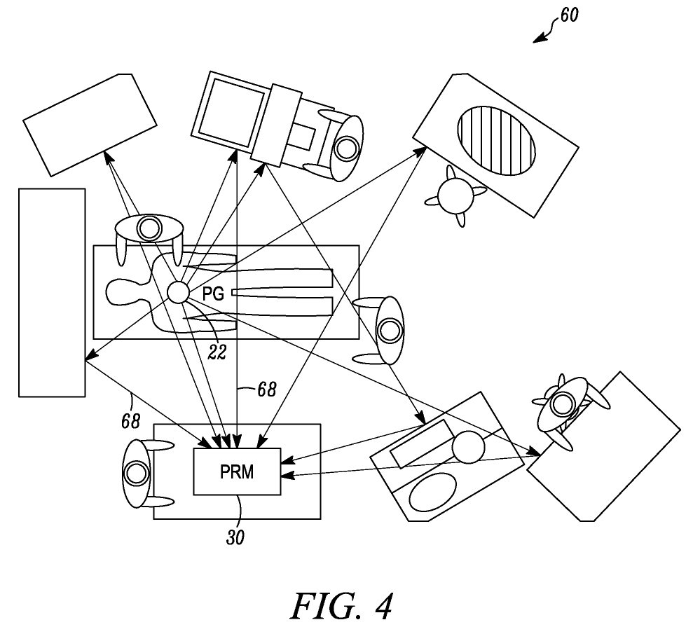

In operation, a signal 21 is transmitted from the antenna 38 of AIMD 22 and is received at the antenna 40 of PRM 30. Retrodirective antenna 40 has the advantage of being able to communicate with AIMD 22 without necessarily having a direct line of sight (LOS) signal transmission path. FIG. 3 depicts the operation of a retrodirective antenna where LOS is available and FIG. 4 depicts the operation of a retrodirective antenna where LOS is not available. As shown in FIG. 3, a patient 24 is located in a surgical suite 60 in medical facility 26. There are a variety of potential signal obstacles 62 present in surgical suite 60, including personnel, equipment, and furniture. Signal obstacles 62 are capable of blocking a direct signal path and are also capable of reflecting signals that are incident upon the obstacle. Patient 24 is depicted as having an AIMD 22 that is either implanted or in the process of being implanted. A PRM 30 is also located in the surgical suite 60. As shown in FIG. 3, there is a direct line of sight 64 from AIMD 22 to PRM 30, where signal 21 is transmitted along direct line of sight 64. There are also signals that are transmitted from AIMD 22 that reflect off of various obstacles 62 and arrive at PRM 30, forming reflected signals 66. However, the signal 64 that has direct line of sight will have the strongest signal power received at the PRM 30 by virtue of the fact that it has traveled the shortest distance and has not reflected off of any other surfaces.

PRM 30 is capable of scanning for the signal with the highest relative signal power and for locking to this signal. For example, the PRM 30 may scan at selected angles within a window from 0 to 180 degrees. The PRM 30 may scan in constant steps (e.g., 1 degree/step). As the PRM 30 scans, the PRM 30 measures signal power or voltage at each scan angle. The PRM 30 stores each angle with a corresponding power measurement. The PRM 30 then locks to the signal with the highest power measurement, and this signal constitutes signal 21.

In an embodiment, AIMD 22 sends a pilot signal until the PRM 30 locks to this pilot signal. After obtaining signal lock on the pilot signal, the PRM 30 sends a command to AIMD 22 that notifies AIMD 22 that signal lock has been obtained and that data can be transmitted. The pilot signal may be transmitted at one frequency, while other data signals may be transmitted at other frequencies.

While LOS is present, as shown in FIG. 3, all signals except the signal on direct line of sight 64 are treated as interference signals by the PRM 30. This situation is referred to as the Rician fade phenomena. The PRM 30 turns off all co-channel and adjacent channel noise and keeps enabled only the channel associated with the direct signal 64. The PRM 30 also turns off all communication channels receiving reflected signals. The PRM 30 maintains the signal lock while obtaining data from the AIMD 22, until something happens to cause the PRM 30 to lose signal lock. This may occur, for example, if a person steps between the AIMD 22 and the PRM 30, such as the situation depicted in FIG. 4, and thereby obstructs the LOS between the AIMD 22 and the PRM 30.

FIG. 4 illustrates the surgical suite of FIG. 3 in which LOS has been lost between the AIMD 22 and the PRM 30. In FIG. 4, a member of the medical staff has moved between the AIMD 22 and the PRM 30, thereby obstructing LOS. The direct signal from the AIMD 22 is no longer available to the PRM 30. Instead, a plurality of reflected signals 68 are received at PRM 30. This situation gives rise to the Rayleigh fade phenomena, in which it is assumed that the power of the other signals (e.g., the reflected signals) will vary randomly according to a Rayleigh distribution.

When LOS is not present, as illustrated in FIG. 4, the strongest reflected signal 68 from the AIMD 22 is used by the PRM 30. When LOS is lost, generally signal lock will be lost by the PRM 30, and this will cause the PRM 30 to rescan for a desired signal. The PRM 30 may command the AIMD 22 to begin transmitting the pilot signal, or the AIMD 22 may already be transmitting the pilot signal, depending on the particular implementation. In any event, the gain of the received pilot signal is compared at each direction during the scan, as described herein. In the illustrated scenario, the PRM 30 locks onto the pilot signal in the direction of the reflected signal, and begins gathering data from the reflected signal. The PRM 30 then ignores the other reflected signals, considering them to be noise.

When a signal is received at the patch antennas 42 within the retrodirective antenna 40, the patches each conjugate the phase (change the phase to its opposite or negative value) and modulates the information on the signal wave. The conjugated signal wave is then amplified and radiated from the patch antennas 42 as signal 43. Conjugating the signal helps to ensure that the signal wave that is radiated from the various patch antennas 42 will collimate precisely at the pilot wave radiating point.

Referring back to FIG. 1, the signal 43 radiated from the patch antennas 42 is transmitted to, and received by, a site repeater 70. Site repeater 70 is typically located at or near the medical facility 26, or is at least located within the range of the signal 43 transmitted from the PRM 30. The purpose of site repeater 70 is to receive the signal 43 transmitted from PRM 30 and to amplify and re-transmit the signal as signal 71 to a local ground station 72. Site repeater 70 is configured to transmit a signal 71 that is in a format or that has a characteristic that is designed to be received and used by local ground station 72.

In one embodiment, local ground station 72 is a component of a conventional satellite communications system. For example, local ground station 72 could be a component of the GlobalStar satellite communications system, Iridium low earth orbit (LEO) satellite communication system, or any other satellite communication system. Local ground station 72 is configured to receive a signal 71 from site repeater 70 and to transmit a corresponding signal 73 to an earth-orbiting satellite 74. In one embodiment, satellite 74 is also a component of a conventional satellite communications system, such as the GlobalStar satellite communications system, Iridium low earth orbit (LEO) satellite communication system, or any other satellite communication system. Satellite 74 may be a single earth-orbiting satellite, or may be one of a network of earth-orbiting satellites. Satellite 74 is configured to receive a signal 73 from local ground station 72 and to transmit a corresponding signal 75 to a remote ground station 76.

Remote ground station 76 is configured to receive signal 75 from satellite 74 and to transmit a corresponding signal 77 to a remote site repeater 78. Remote ground station 76 is generally constructed in a similar manner to local ground station 72. Remote ground station 76 may also be a component of a conventional satellite communications system, such as the GlobalStar satellite communications system, Iridium low earth orbit (LEO) satellite communication system, or any other satellite communication system. Signal 77 is received at remote site repeater 78 and re-transmitted as signal 79. Signal 79 is received at remote device 80. Remote device 80 is generally configured to receive signal 79 and to process signal 79 and/or to provide an interface to signal 79. There are a number of usable embodiments of remote device 80. For example, remote device 80 may be a PRM device similar to PRM 30 that allows the data encoded in signal 79 to be recorded or monitored. Because each signal transmission in communication system 20 is bidirectional, remote device 80 may also be a programmer or have device programming capabilities. In one embodiment, a physician at remote location 28 uses remote device 80 to receive a signal from AIMD 22 and to perceive the information that is encoded in the signal. The physician may also act in response to the information in the received signal, such as by selecting a different manner of control for the AIMD 22 and then initiating a signal from the remote device 80 that propagates through the communications system 20 back to AIMD 22. In this way, a physician at a remote location has the ability to monitor a patient and to deliver a medical therapy to a patient, even where traditional means of communication such as phone lines or the Internet are not available.

There are a number of usable embodiments of signals 21, 43, 71, 73, 75, 77, 79 within communication system 20. In one usable embodiment, each of signals 21, 43, 71, 73, 75, 77, 79 are at the same frequency. In another usable embodiment, signals 21, 43, 71, 73, 75, 77, 79 are at different frequencies. In some embodiments, some of signals 21, 43, 71, 73, 75, 77, 79 are at the same and some are at different frequencies. By way of example, it may be desired that signal 21 be at a different frequency than the other signals because of the special needs of transmitting a signal out of a human body. The frequency of signal 21 generally must be tested for its effect on tissue heating and other possibly undesirable effects. The optimal or desired frequency chosen for signal 21 may not necessarily be the optimal or desired frequency for other transmissions within the system.

In one embodiment, some or all of signals 21, 43, 71, 73, 75, 77, 79 are high frequency signals. In one embodiment, some or all of the transmission signals 21, 43, 71, 73, 75, 77, 79 are at a frequency equal to or greater than 1 GHz. In another embodiment, some or all of the transmission signals 21, 43, 71, 73, 75, 77, 79 are equal to or greater than 2 GHz. In a further embodiment, some or all of the transmission signals are at 2.4 to 2.5 GHz. In yet another embodiment, some or all of the transmission signals 21, 43, 71, 73, 75, 77, 79 are 2.4835 to 2.5 GHz. In some embodiments, some or all of the transmission signals 21, 43, 71, 73, 75, 77, 79 are in the S-band of about 2 to 4 GHz. In some other embodiments, some or all of the transmission signals 21, 43, 71, 73, 75, 77, 79 are in the C-band of about 4 to 8 GHz. In yet other embodiments, some or all of the transmission signals 21, 43, 71, 73, 75, 77, 79 are in the X-band of about 8 to 12 GHz.

A further advantage of using frequencies in the X-band of about 8 to 12 GHz is the relatively lower probability of interference. There are relatively fewer wireless devices in use that operate in this frequency spectrum than in other frequency spectrums. Furthermore, the allocation of usage of this band tends to be currently less congested and less utilized in most countries of the world, although this is subject to change. Some current medical device communication systems operate in different bands that tend to be much more congested and fully utilized around the world. This situation may lead to a communication system that can only work in one country because the operating frequency is not available for use in other countries. By operating in an X-band spectrum, it is expected that a single frequency can be utilized in most or all countries of the world, greatly promoting portability and interchangeability of the medical communication system.

An alternative embodiment of a portion of medical communications system 20 is depicted in FIG. 7. The portion of medical communication system 120 depicted in FIG. 7 relates to the transmission of a signal from an AIMD 22 to a PRM 130. PRM 130 is generally similar to PRM 30 described above. Patient 24 has an AIMD 22 that transmits a signal 125. Whereas in system 20 the signal from the AIMD 22 is received directly in PRM 130, in system 120 the signal 125 from AIMD 22 is received at a wand 126 that is positioned near patient 24. There are at least two usable embodiments of wand 126. In one usable embodiment, wand 126 has a wired connection to PRM 130 and wand 126 is configured to transmit a signal corresponding to the signal received from AIMD 22 across the wired connection. In another usable embodiment, wand 126 is configured to transmit a wireless signal to PRM 130. Wand 126 includes an antenna 128. In one embodiment, antenna 128 is a retrodirective antenna that operates in the same manner as the retrodirective antenna 40 described above. Wand 126 serves to provide a location close to AIMD 22 to receive the signal from the AIMD 22. This is advantageous because the signal transmitted from the AIMD 22 is often at relatively low power in order to minimize the risk of tissue heating and also to conserve the available battery power in the implantable device. The wand 126 is then configured to transmit the signal on to the PRM 130 in a manner that is analogous to the operation of PRM 130.

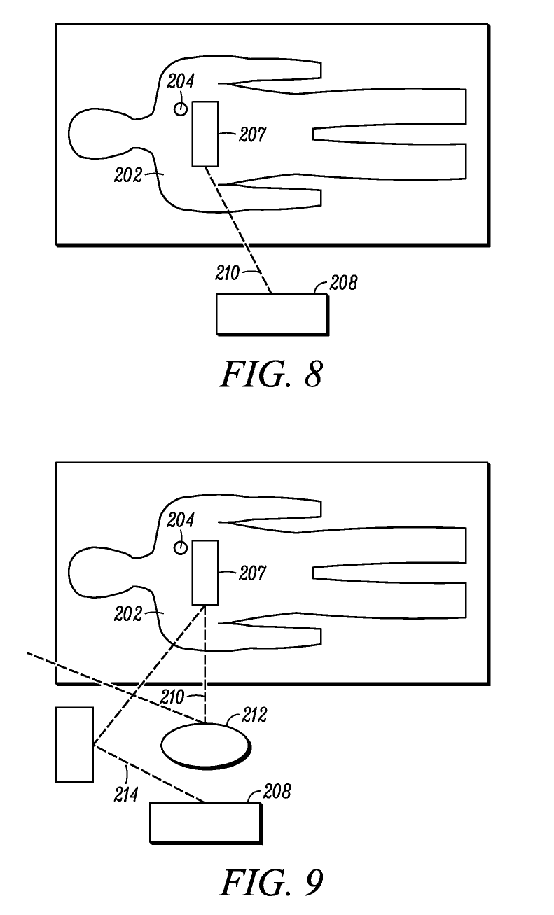

Yet another embodiment of the invention is depicted in FIG. 8. The embodiment of FIG. 8 includes a wireless wand for transmitting signals to a programmer. In the embodiment of FIG. 8, a patient 202 has a medical device, such as a pulse generator 204, implanted in the patient's upper chest. The pulse generator 204 is in communication with a wand 207, which is in communication with external programmer 208. The wand 207 includes an antenna that is operable to emit signals in an omni radiation pattern. The wand 207 transmits a pilot signal to initiate communication with the programmer 208.

The programmer 208 includes multiple antennas, such as a phased array of patch antennas, which receive the signal from the wand. In this implementation, the programmer 208 obtains signal lock with the wand by scanning for the signal with the highest relative power. To illustrate, assume an axis extends out from the base of the programmer 208, and parallel to the back face of the programmer 208. The programmer 208 scans at selected angles from the axis from 0 degrees to 180 degrees. The programmer may scan in constant steps (e.g., 1 degree per step). As the programmer 208 scans, the programmer 208 measures signal power or voltage at each scan angle. The programmer 208 stores each angle with a corresponding power measurement.

As shown in FIG. 8, there are no obstructions between the wand and the programmer 208. As such, the programmer is in line of sight (LOS) of the wand 207, and the programmer 208 determines that the direct signal 210 obtained in the LOS direction has the best signal power. Therefore, the programmer 208 locks on to the direct signal 210 from the wand 207. After obtaining signal lock on the pilot signal, the programmer 208 sends a command to the wand that notifies the wand 207 that signal lock has been obtained, and that data from the pulse generator 204 can be transmitted. The pilot signal may be transmitted at one frequency, while other data signals may be transmitted at other frequencies.

While LOS is present, all signals except the direct signal 210 are treated as interference signals by the programmer 208. This situation is referred to as the Rician fade phenomena. The programmer 208 turns off all co-channel and adjacent channel noise and keeps enabled only the channel associated with the direct signal 210. The programmer 208 also turns off all communication channels receiving reflected signals.

The programmer 208 maintains lock while obtaining data from the wand, until something happens to cause the programmer 208 to lose signal lock. This may occur, for example, if a person steps in between the wand 207 and the programmer 208, and thereby obstructs the LOS between the wand 207 and the programmer 208.

FIG. 9 illustrates a plan view in which LOS has been lost between the wand 207 and programmer 208. In FIG. 9, an obstruction 212, such as a member of a medical staff has moved between the wand 207 and programmer 208, thereby obstructing LOS. The formerly-direct signal 210 from the wand 207 is no longer available to the programmer 208. This situation gives rise to the Rayleigh fade phenomena, in which it is assumed that the power of the other signals (e.g., the reflected signals) will vary randomly according to a Rayleigh distribution.

When LOS is not present, as illustrated in FIG. 9, the strongest reflected signal 214 from the wand 207 is used by the programmer 208. When LOS is lost, generally signal lock will be lost by the programmer 208, and thus will cause the programmer 208 to rescan for a desired signal. The programmer 208 may command the wand to begin transmitting the pilot signal, or the wand may already be transmitting the pilot signal, depending on the particular implementation. In any event, the gain of the received pilot signal is compared at each direction during the scan. In the circumstance shown in FIG. 9, the programmer 208 locks onto the pilot signal in the direction of the reflected signal 214, and begins gathering data from the reflected signal 214. The programmer 208 ignores the other reflected signals, considering them to be noise.

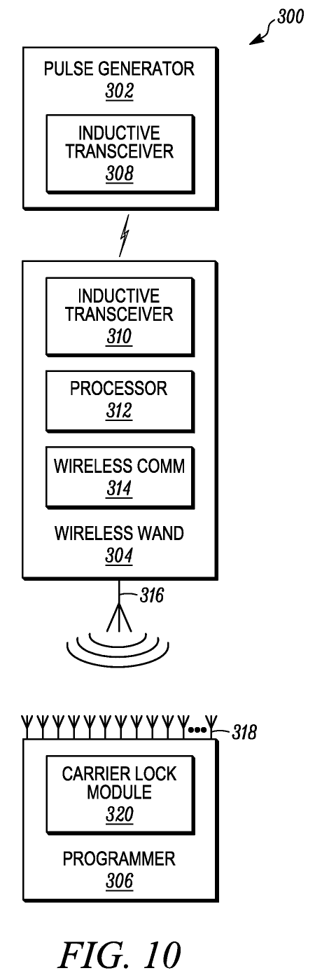

FIG. 10 is a block diagram illustrating functional modules in a wireless wand system 300 according to one embodiment of the invention. As used herein, a module represents a software, hardware, or firmware component (or any combination thereof). The wireless wand system 300 includes an implantable medical device (IMD), such as a pulse generator 302 in communication with a wand 304, which is in communication with an external programmer 306.

In general, the wand 304 wirelessly emits a signal that the programmer 306 can detect in order to establish signal lock with the wand 304. After signal lock is achieved, the wand 304 receives data from the pulse generator 302 and emits a signal or signals including the medical data received from the pulse generator 302.

More specifically, the pulse generator 302 includes an inductive transceiver module 308 and the wand 304 includes an inductive transceiver module 310. Via the inductive transceiver modules 308 and 310, the pulse generator 302 and the wand 304 communicate with each other. The inductive communication between the pulse generator 302 and the wand 304 is referred to as magnetic or near field communication. The pulse generator 302 communicates various types of data to the wand 304 including, but not limited to, sensor data, e-gram data, status data, and timing data. The pulse generator 302 may communicate a specified type of data in response to a request from the programmer 306.

The wand 304 includes a processor 312 that communicates with other components of the wand 304 to control the wand's operation. The processor 312 may be any of various types of processor, including but not limited to, a microprocessor, a microcontroller, a digital signal processor, or an application specific integrated circuit (ASIC). The wand 304 also includes a wireless communication module 314 for communication with the programmer 306.

The wireless communication module 314 generates a signal via antenna 316 of the wand 304. The wand antenna 316 is a dipole antenna and is operable to transmit signals in an omni radiation pattern (or substantially omni radiation pattern). The wireless communication module 314 can communicate using any of various types of wireless modes, including, but not limited to, radio frequency (RF) or microwave. Signals transmitted by the wireless communication module 314 can be modulated using any of various modulation techniques, such as amplitude shift keying (ASK, e.g., on-off keying (OOK)), binary phase-shift keying (BPSK), and quadrature phase-shift keying (QPSK).

The programmer 306 includes a phased array of antenna elements 318. The elements 318 are typically patch antennas that are light weight, and can be mounted on a flat surface of the programmer 306. Each patch antenna generally has a range of 0 to 6 dB in gain. Conformal mapping to the surface of the programmer 306 can be used to take up less area and minimize volume of programmer 306. Patch antennas are easily combined to form arrays to provide for higher gain. In addition, patch antennas are typically less expensive than other types of antennas.

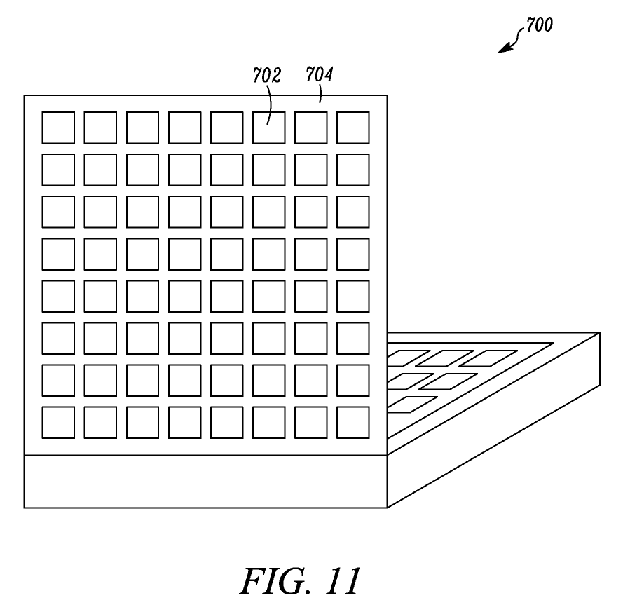

One embodiment of a programmer 700 is illustrated in FIG. 11. Programmer 700 includes an array of sixty-four patch antennas 702 mounted on the top cover 704 of the programmer 700.

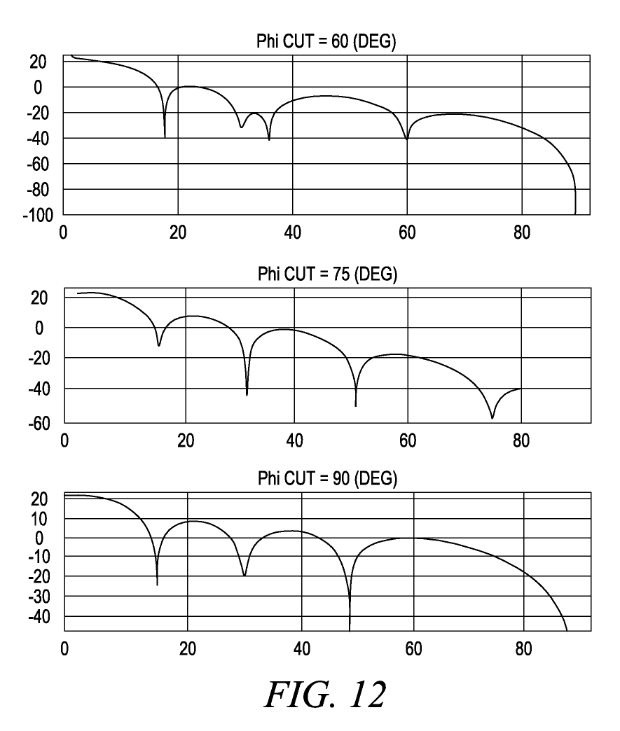

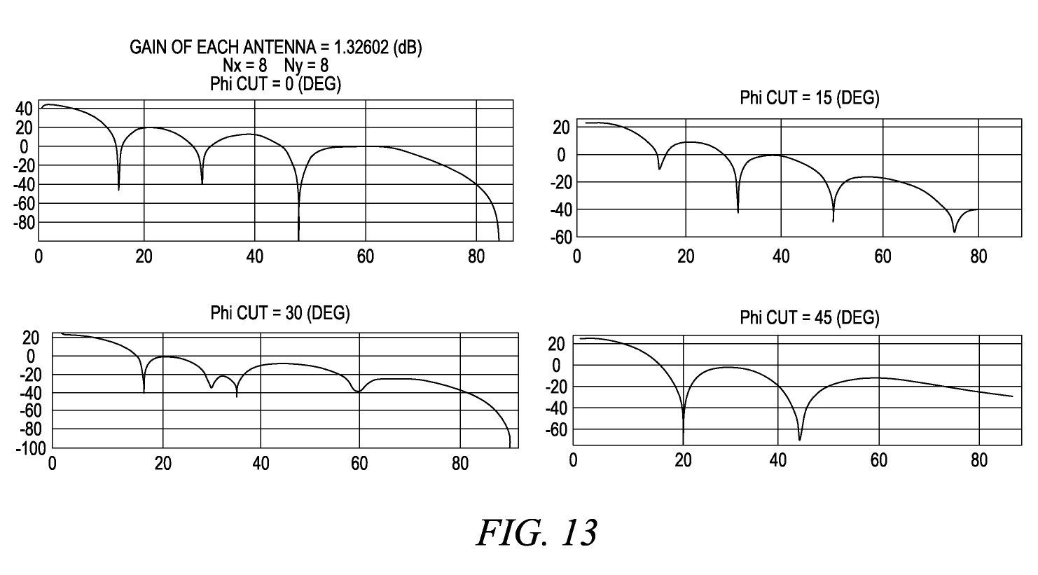

The antenna 316 of wireless wand 304 is a dipole antenna that is operable to generate signals in an approximately omni-directional radiation pattern. Depending on the length of the antenna compared to the frequency it is using, the radiated pattern may have multiple lobes. The radiated signal either goes directly to the receiving array that is mounted on the back panel of the programmer or it is reflected by the surroundings and the reflected signal reaches the array. FIGS. 12-13 show the power pattern of the array antenna that has an 8×8 array of patches. The plots are shown in two dimensions; however, in actual operation the lobes would be three-dimensional. The central lobe always provides the maximum amount of gain, while the side lobes provide gradually decreasing gain. In effect the higher gain of the main lobes provides the necessary discrimination between the direct (strongest) signal and the reflected (weaker) signals. The programmer 306 includes a carrier lock module 320 that performs a carrier lock algorithm for locking onto the carrier of signals transmitted by the wand.

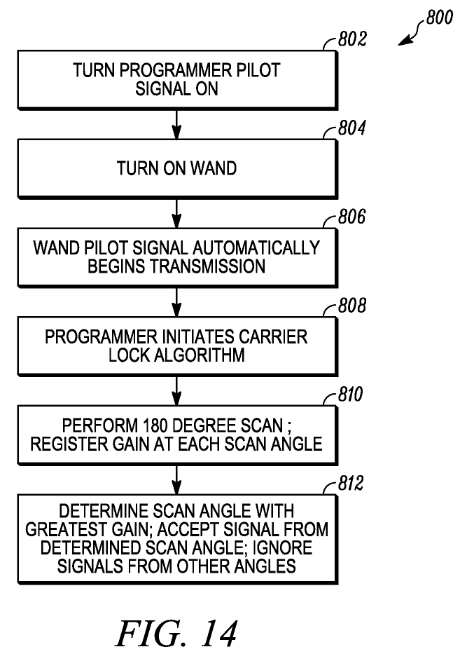

FIG. 14 is a flow chart illustrating an algorithm 800 that may be carried out by a programmer for wireless communication with a wand or an implantable medical device. The algorithm is generally the same whether the programmer is communicating directly with a wand or directly with an implantable medical device. For ease of description, the algorithm will be described with respect to the operation with the wand, although it will be appreciated that the algorithm is adaptable to use directly with an implantable medical device. The programmer pilot signal is turned on at step 802. At step 804 the wand is enabled. The wand's pilot signal begins being transmitted automatically at step 806. At step 808, the programmer initiates a carrier lock algorithm using the array of patch antennas. To determine the direction of greatest signal power, the programmer performs a 180 degree scan at step 810. The programmer steps through angles through 180 degrees and registers the detected power at each scan angle. At step 812, the programmer analyzes the registered power values and chooses the scan angle with the greatest power, and accepts the wand signal from the chosen direction, while ignoring signals from other directions. As described herein, the selected signal may be a direct signal, directly received from the wand, or the selected signal may be a reflected signal that has reflected off objects in the operating room.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

The above specification provides a complete description of the structure and use of the invention. Since many of the embodiments of the invention can be made without parting from the spirit and scope of the invention, the invention resides in the claims.

What is claimed is: where the programmer locks to the signal having the highest signal power.

1. A medical communication system comprising:
(i) an active implantable medical device (AIMD) configured to transmit and receive wireless signals from within a human body;
(ii) a non-implantable programmer including a retrodirective antenna, the programmer being configured to scan in multiple directions for signals received from the AIMD and to identify the direction of the signal having the highest signal power.

2. The medical communication system of claim 1, where the non-implantable programmer is further configured to:
(a) receive a wireless signal from the AIMD; and
(b) conjugate the phase of the received signal, amplify the conjugated signal, and radiate the amplified signal.

3. The medical communication system of claim 1, where the retrodirective antenna comprises an aperture coupled antenna.

4. The medical communication system of claim 1, where the retrodirective antenna comprises a circularly polarized antenna.

5. The medical communication system of claim 1, where the retrodirective antenna comprises a patch antenna.

6. The medical communications system of claim 1, where the programmer is located within 3 meters of the AIMD.

7. The medical communications system of claim 1, where the programmer is located more than 3 meters from the AIMD.

8. The medical communication system of claim 2, where the radiated amplified signal is received at a site repeater.

9. The medical communications system of claim 8, where the site repeater is configured to communicate with a satellite network.

10. The medical communications system of claim 2, where the radiated amplified signal is received by a remote electrical device.

11. The medical communications system of claim 10, where the programmer is further configured to receive a wireless signal from the remote electrical device and to transmit a corresponding wireless signal to the AIMD.

12. The medical communications system of claim 1, where the programmer is further configured to receive a wireless signal from more than one AIMD.

13. The medical communications system of claim 1, where the wireless signal from the AIMD has a frequency of 2 to 4 GHz.

14. The medical communications system of claim 1, where the wireless signal from the AIMD has a frequency of 4 to 8 GHz.

15. The medical communications system of claim 1, where the wireless signal from the AIMD has a frequency of 8 to 12 GHz.

16. A medical communication system comprising:
(i) an active implantable medical device (AIMD) configured to transmit a wireless signal;
(ii) a programmer having a retrodirective antenna that is configured to receive the wireless signal from the AIMD and to transmit a corresponding signal;
(iii) a local site repeater configured to receive the signal from the programmer and to transmit a corresponding signal;
(iv) a local ground station configured to receive the signal from the site repeater and to transmit a corresponding signal;
(v) a space-based satellite configured to receive the signal from the local ground station and to transmit a corresponding signal to a remote ground station;
(vi) the remote ground station configured to receive the signal from the space-based satellite and to transmit a corresponding signal;
(vii) a remote site repeater configured to receive the signal from the remote ground station and to transmit a corresponding signal; and
(viii) a remote device configured to receive the signal from the remote site repeater and to provide an interface to the signal.

17. The medical communication system of claim 16, where the wireless signal transmitted by the AIMD has a frequency of 1.5 to 5.2 GHz.

18. The medical communication system of claim 16, where the wireless signal transmitted by the AIMD has a frequency of 5.2 to 10.9 GHz.

19. The medical communication system of claim 16, where each signal in the system has the same frequency.

20. The medical communications system of claim 16, where each signal in the system is not at the same frequency.

21. The medical communication system of claim 16, further configured to transmit a signal from the remote device to the AIMD.

22. The medical communications system of claim 21, where the signal transmitted from the remote device to the AIMD causes a medical therapy to be administered to a patient.
